(19) United States
(12) Patent Application Publication
(10) Pub. No.: US 2021/0309645 A1
(43) Pub. Date: Oct. 7, 2021

(54) pH DEPENDENT PHOTOACOUSTIC COMPOUNDS AND APPLICATIONS THEREOF

(71) Applicant: Wake Forest University, Winston-Salem, NC (US)

(72) Inventor: Lacey McNally, East Bend, NC (US)

(21) Appl. No.: 17/265,978

(22) PCT Filed: Aug. 6, 2019
(86) PCT No.: PCT/US2019/045258

§ 371 Date: Feb. 4, 2021

Related U.S. Application Data
(60) Provisional application No. 62/715,550, filed on Aug. 7, 2018.

(51) Int. Cl. C07D 405/06; A61B 5/00; A61K 49/22

(52) U.S. Cl. CPC C07D 405/06; A61K 49/22; A61B 5/0095

(57) ABSTRACT

In one aspect, photoacoustic compounds are described herein. A photoacoustic compound, for example, comprises two multicyclic ring moieties coupled by a rotationally restricted linkage, wherein the photoacoustic compound exhibits at least one of a pH dependent absorption spectrum, pH dependent emission spectrum and pH dependent photoacoustic spectrum. In being pH dependent, the absorption, emission and/or photoacoustic spectra of the photoacoustic compound can vary in response to environmental pH changes. Accordingly, the photoacoustic compound can provide differing photoacoustic responses based on the local pH environment.

### RELATED APPLICATION DATA

The present application claims priority to U.S. Provisional Patent Application Ser. No. 62/715,550 filed Aug. 7, 2018, which is incorporated herein by reference in its entirety.

### FIELD

The present application relates to photoacoustic compounds and, in particular, to photoacoustic compounds having pH dependent absorption spectra.

### BACKGROUND

While near infrared (NIR) fluorescent intraoperative imaging systems are able to provide simultaneous acquisition of surgical anatomy and NIR fluorescence signal, two major impediments, limited depth of resolution (5-8 mm) below the surface and limited cancer-specific probes with sufficient signal, restrict the potential for image-guided surgical resection to more superficial tumors, i.e. melanoma, some superficial head and neck squamous cell carcinoma, other oral cancers, and some thyroid or breast cancers. While intraoperative imaging strategies to visualize cancer cells accurately are necessary, it is critical to develop intraoperative imaging methods that can detect molecular information at depths of centimeters for non-superficial cancers, while retaining high resolution and tumor specificity.

Photoacoustic (optoacoustic) imaging is emerging to drive optical imaging beyond the penetration limits of conventional methods by allowing the formation of optical images several centimeters inside tissue. Multispectral optoacoustic imaging is based on the optoacoustic effect: the conversion of absorbed electromagnetic energy (e.g. NIR light) to acoustic signals. The selective absorption of light at multiple wavelengths and slices enables 3D volumetric spectrally enriched (color) imaging from deep living tissues in real time and at high spatial resolution. MSOT imaging operates through centimeters of tissue enabling tomographic 3D imaging with optical contrast at depths of ultrasound, in real-time. Currently, clinical Multispectral Optoacoustic imaging systems are in testing to improve tumor identification in Europe and recently the United States. To maximize the potential of clinical MSOT imaging, tumor specific contrast agents must be developed.

### SUMMARY

In one aspect, photoacoustic compounds or optoacoustic compounds are described herein. Photoacoustic and optoacoustic are generally used interchangeably in the art. A photoacoustic compound, for example, comprises two multicyclic ring moieties coupled by a rotationally restricted linkage, wherein the photoacoustic compound exhibits at least one of a pH dependent absorption spectrum, pH dependent emission spectrum and pH dependent photoacoustic spectrum. In being pH dependent, the absorption, emission and/or photoacoustic spectra of the photoacoustic compound can vary in response to environmental pH changes. Accordingly, the photoacoustic compound can provide differing photoacoustic responses based on the local pH environment. In this way, various targets can be imaged and resolved based on pH variance between the targets.

In some embodiments, a photoacoustic compound described herein is of Formula (I):

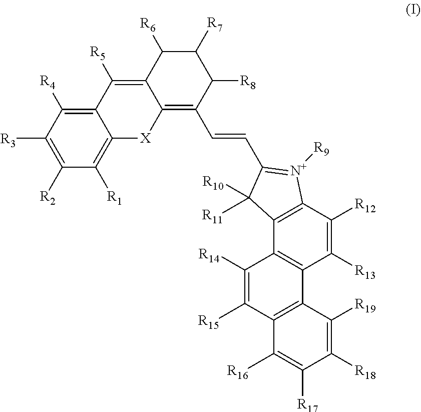

wherein R₁-R₁₉ are independently selected from the group consisting of hydrogen, alkyl, alkenyl, heteroalkyl, cycloalkyl, heterocycloalkyl, aryl, heteroaryl, alkyl-aryl, alkyl-heteroaryl, alkoxy, hydroxyl, halo, and amide, wherein the alkyl, alkenyl, heteroalkyl, cycloalkyl, heterocycloalkyl, aryl, heteroaryl, alkyl-aryl, alkyl-heteroaryl, alkoxy, and amide are optionally substituted with one or more substituents selected from the group consisting of (C₁-C₁₀)-alkyl, (C₁-C₁₀)-alkenyl, heteroalkyl, cycloalkyl, heterocycloalkyl, aryl, heteroaryl, alkoxy, amide, halo, hydroxy, C(O)OR₂₀, and C(O)R₂₁, wherein R₂₀ is selected from the group consisting of hydrogen, alkyl and alkenyl and R₂₁ is selected from the group consisting of hydrogen, alkyl, alkenyl, heteroalkyl, cycloalkyl, heterocycloalkyl, aryl, heteroaryl and NR₂₂R₂₃, wherein Rn and R₂₃ are independently selected from the group consisting of hydrogen, alkyl, alkenyl, aryl and heteroaryl; and wherein X is selected from the group consisting of CR₂₄R₂₅, N and O, wherein R₂₄ and R₂₅ are independent selected from the group consisting of hydrogen, alkyl, alkenyl, hydroxyl and halo.

In some embodiments, a photoacoustic compound described herein is of Formula (II):

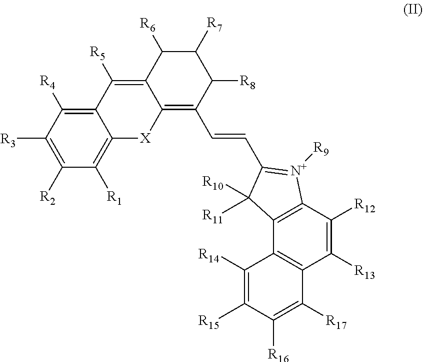

wherein R₁-R₁₇ are independently selected from the group consisting of hydrogen, alkyl, alkenyl, heteroalkyl, cycloalkyl, heterocycloalkyl, aryl, heteroaryl, alkyl-aryl, alkyl-heteroaryl, alkoxy, hydroxyl, halo, and amide, wherein the alkyl, alkenyl, heteroalkyl, cycloalkyl, heterocycloalkyl, aryl, heteroaryl, alkyl-aryl, alkyl-heteroaryl, alkoxy, and amide are optionally substituted with one or more substituents selected from the group consisting of (C₁-C₁₀)-alkyl, (C₁-C₁₀)-alkenyl, heteroalkyl, cycloalkyl, heterocycloalkyl, aryl, heteroaryl, alkoxy, amide, halo, hydroxy, C(O)OR₁₈, and C(O)R₁₉, wherein Rig is selected from the group consisting of hydrogen, alkyl and alkenyl and Rig is selected from the group consisting of hydrogen, alkyl, alkenyl, heteroalkyl, cycloalkyl, heterocycloalkyl, aryl, heteroaryl and NR₂₀R₂₁, wherein R₂₀ and R₂₁ are independently selected from the group consisting of hydrogen, alkyl, alkenyl, aryl and heteroaryl; and wherein X is selected from the group consisting of CR₂₂R₂₃, N and O, wherein R₂₄ and R₂₅ are independent selected from the group consisting of hydrogen, alkyl, alkenyl, hydroxyl and halo.

In another aspect, methods of imaging are described. Briefly, a method of imaging a target comprises disposing a photoacoustic compound in an environment adjacent to the target and irradiating the photoacoustic compound with one or more wavelengths of electromagnetic radiation. Soundwaves produced by the photoacoustic compound in response to the electromagnetic radiation are detected and transformed into an image. The photoacoustic compound comprises two mutlicyclic ring moieties coupled by a rotationally restricted linkage, and exhibits at least one of a pH dependent absorption spectrum, pH dependent emission spectrum and pH dependent photoacoustic spectrum. In some embodiments, the photoacoustic compound is irradiated with multiple wavelengths of radiation resulting in multiple wavelength dependent images of the target. As described further herein, each of the wavelength dependent images can correspond to the photoacoustic compound at locations of differing pH in the environment. The wavelength dependent images can be overlaid to present a detailed image of the target.

These and other embodiments are described in greater detail in the following detailed description.

### BRIEF DESCRIPTION OF THE DRAWINGS

FIG. 1 illustrates several non-limiting embodiments of photoacoustic compounds of Formula (I) and the associated pH-driven zwitterionic behavior of the compounds.

FIG. 2 illustrates a zwitterionic compound of Formula (I) according to one non-limiting embodiment.

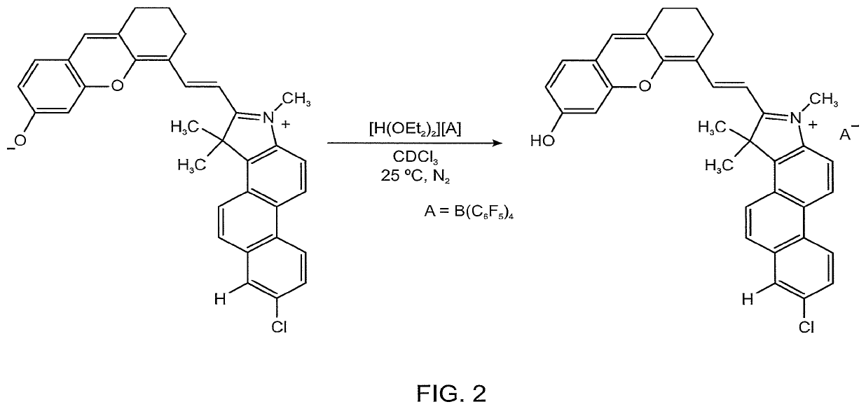

FIGS. 3(A-D) illustrate absorption spectrum modulation of a compound of Formula (I) according to changes in pH in some embodiments.

FIG. 4 illustrates non-responsiveness of the absorption spectrum of a compound of Formula (I) to various ionic species according to some embodiments.

FIG. 5 illustrates specific identification via MSOT of acidic pHe within an orthotopic pancreatic tumor by a compound of Formula (I) according to some embodiments.

FIG. 6 illustrates 3D accumulation of a compound of Formula (I) within an orthotopic pancreatic tumor according to some embodiments.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### Definitions

The term “alkyl” as used herein, alone or in combination, refers to a straight or branched saturated hydrocarbon group optionally substituted with one or more substituents. For example, an alkyl can be C₁-C₃₀ or C₁-C₁₈.

The term “alkenyl” as used herein, alone or in combination, refers to a straight or branched chain hydrocarbon group having at least one carbon-carbon double bond and optionally substituted with one or more substituents.

The term “aryl” as used herein, alone or in combination, refers to an aromatic monocyclic or multicyclic ring system optionally substituted with one or more ring substituents.

The term “heteroaryl” as used herein, alone or in combination, refers to an aromatic monocyclic or multicyclic ring system in which one or more of the ring atoms is an element other than carbon, such as nitrogen, oxygen and/or sulfur.

The term “cycloalkyl” as used herein, alone or in combination, refers to a non-aromatic, mono- or multicyclic ring system optionally substituted with one or more ring substituents.

The term “heterocycloalkyl” as used herein, alone or in combination, refers to a non-aromatic, mono- or multicyclic ring system in which one or more of the atoms in the ring system is an element other than carbon, such as nitrogen, oxygen or sulfur, alone or in combination, and wherein the ring system is optionally substituted with one or more ring substituents.

The term “heteroalkyl” as used herein, alone or in combination, refers to an alkyl moiety as defined above, having one or more carbon atoms in the chain, for example one, two or three carbon atoms, replaced with one or more heteroatoms, which may be the same or different, where the point of attachment to the remainder of the molecule is through a carbon atom of the heteroalkyl radical.

The term “alkoxy” as used herein, alone or in combination, refers to the moiety RO—, where R is alkyl or alkenyl defined above.

The term “halo” as used herein, alone or in combination, refers to elements of Group VIIA of the Periodic Table (halogens). Depending on chemical environment, halo can be in a neutral or anionic state.

### I. Photoacoustic Compounds

In one aspect, photoacoustic compounds are described herein. A photoacoustic compound, for example, comprises two multicyclic ring moieties coupled by a rotationally restricted linkage, wherein the photoacoustic compound exhibits at least one of a pH dependent absorption spectrum, pH dependent emission spectrum and pH dependent photoacoustic spectrum. In being pH dependent, the absorption, emission and/or photoacoustic spectra of the photoacoustic compound can vary in response to environmental pH changes. Accordingly, the photoacoustic compound can provide differing photoacoustic responses based on the local pH environment.

As detailed further herein, photoacoustic compounds can be zwitterionic. In some embodiments, the two multicyclic ring moieties are oppositely charged. Alternatively, one of the two multicyclic ring moieties can possess both positive and negatively charged groups. Moreover, each of the multicyclic ring moieties can exhibit conjugation and/or aromaticity. In some embodiments, at least one of the multicyclic ring moieties is aromatic. In such embodiments, the other multicyclic moiety coupled to the rotationally restricted linkage is also aromatic or partially conjugated. The rotationally restricted linkage between the two multicylic moieties can render the photoacoustic compound planar or substantially planar. However, the photoacoustic compound can exhibit out of plane flexing. In some embodiments, the photoacoustic compound exhibits out of plane flexing in response to exposure to electromagnetic radiation of one or more wavelengths. The rotationally restricted linkage between the multicyclic moieties can comprise an unsaturated hydrocarbon, in some embodiments. The unsaturated hydrocarbon can be linear, branched or cyclic and can contain any desired number of unsaturation points. Additionally, the unsaturated hydrocarbon can exhibit conjugation. In some embodiments, the rotationally restricted linkage establishes conjugation between the two multicyclic ring moieties. One or both of the multicyclic moieties may comprise one or more heteroatoms, such as nitrogen, sulfur and/or oxygen.

Photoacoustic compounds described herein exhibit an absorption spectrum, emission spectrum and/or photoacoustic spectrum that is pH dependent. In some embodiments, absorption and emission maxima blueshift in response to decreasing pH value. Alternatively, absorption and emission maxima can redshift in response to decreasing pH value. Redshift or blueshift of absorption and emission maxima in response to increasing or decreasing pH values can be dependent on the specific compositional and structural parameters of the photoacoustic compounds. Photoacoustic compounds can be highly sensitive to changes in pH. In some embodiments, the absorption, emission and/or photoacoustic spectra of a photoacoustic compound can change in response to a change in pH value 0.1 or greater. For example, the absorption, emission and/or photoacoustic spectra of a photoacoustic compound can change in response a change in pH value of 0.1 to 0.3 or 0.1 to 0.2. Variation of photoacoustic compound spectra can result from protonation and/or deprotonation of the photoacoustic compound in response to pH change.

Additionally, absorption, emission and/or photoacoustic spectra of photoacoustic compounds can be non-responsive to other species including, but not limited to, ions of one or more alkali metals, alkaline earth metals and/or transition metals. For example, one or more spectra of a photoacoustic compound can be non-responsive to salts of Na⁺, K⁺, Ca²⁺, Fe³⁺, Fe²⁺, Mg²⁺ and/or Zn²⁺. In being non-responsive to various metal ionic species, photoacoustic compounds can be employed in a variety of biological and physiological environments for imaging applications based on local pH changes in the environment. Moreover, in some embodiments, photoacoustic compounds described herein are not cytotoxic, thereby further enhancing suitability of biological and physiological environments.

In some embodiments, a photoacoustic compound is of Formula (I):

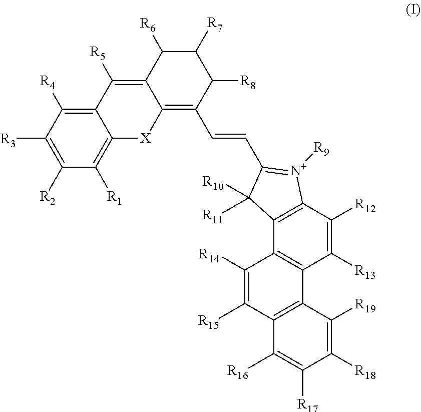

wherein R₁-R₁₉ are independently selected from the group consisting of hydrogen, alkyl, alkenyl, heteroalkyl, cycloalkyl, heterocycloalkyl, aryl, heteroaryl, alkyl-aryl, alkyl-heteroaryl, alkoxy, hydroxyl, halo, and amide, wherein the alkyl, alkenyl, heteroalkyl, cycloalkyl, heterocycloalkyl, aryl, heteroaryl, alkyl-aryl, alkyl-heteroaryl, alkoxy, and amide are optionally substituted with one or more substituents selected from the group consisting of (C₁-C₁₀)-alkyl, (C₁-C₁₀)-alkenyl, heteroalkyl, cycloalkyl, heterocycloalkyl, aryl, heteroaryl, alkoxy, amide, halo, hydroxy, C(O)OR₂₀, and C(O)R₂₁, wherein R₂₀ is selected from the group consisting of hydrogen, alkyl and alkenyl and R₂₁ is selected from the group consisting of hydrogen, alkyl, alkenyl, heteroalkyl, cycloalkyl, heterocycloalkyl, aryl, heteroaryl and NR₂₂R₂₃, wherein R₂₂ and R₂₃ are independently selected from the group consisting of hydrogen, alkyl, alkenyl, aryl and heteroaryl; and wherein X is selected from the group consisting of CR₂₄R₂₅, N and O, wherein R₂₄ and R₂₅ are independent selected from the group consisting of hydrogen, alkyl, alkenyl, hydroxyl and halo.

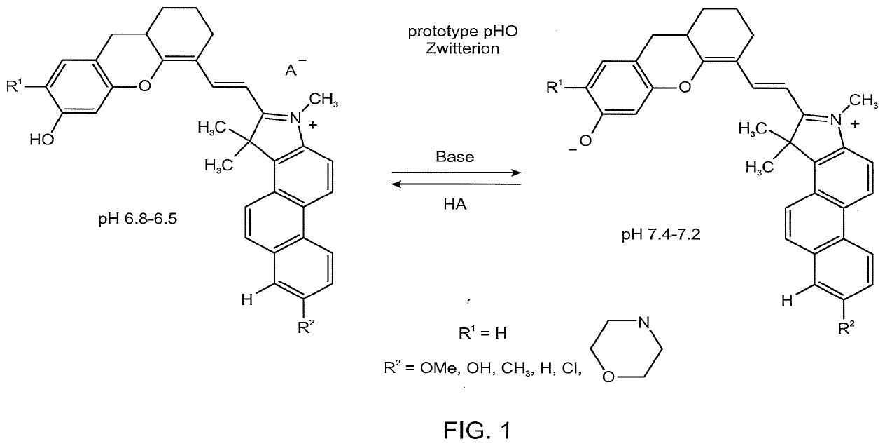

In some embodiments, R₁-R₈ and R₁₂-R₁₉ are independently selected from the group consisting of hydrogen, alkyl, cycloalkyl, heteroalkyl, heterocycloalkyl, alkoxy, hydroxyl, and halo and wherein R₉-R₁₁ are independently selected from alkyl and alkenyl. FIG. 1 illustrates several non-limiting embodiments of photoacoustic compounds of Formula (I) and the associated pH-driven zwitterionic behavior of the compounds.

In some embodiments, a photoacoustic compound is of Formula (II):

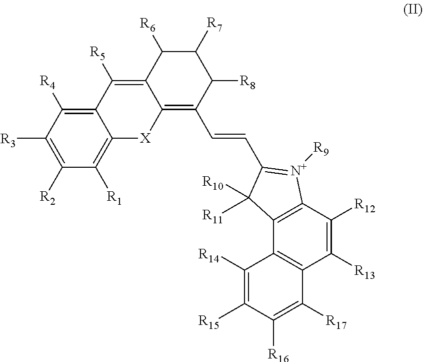

wherein R₁-R₁₇ are independently selected from the group consisting of hydrogen, alkyl, alkenyl, heteroalkyl, cycloalkyl, heterocycloalkyl, aryl, heteroaryl, alkyl-aryl, alkyl-heteroaryl, alkoxy, hydroxyl, halo, and amide, wherein the alkyl, alkenyl, heteroalkyl, cycloalkyl, heterocycloalkyl, aryl, heteroaryl, alkyl-aryl, alkyl-heteroaryl, alkoxy, and amide are optionally substituted with one or more substituents selected from the group consisting of (C₁-C₁₀)-alkyl, (C₁-C₁₀)-alkenyl, heteroalkyl, cycloalkyl, heterocycloalkyl, aryl, heteroaryl, alkoxy, amide, halo, hydroxy, C(O)OR₁₈, and C(O)R₁₉, wherein R₁₈ is selected from the group consisting of hydrogen, alkyl and alkenyl and R₁₉ is selected from the group consisting of hydrogen, alkyl, alkenyl, heteroalkyl, cycloalkyl, heterocycloalkyl, aryl, heteroaryl and NR₂₀R₂₁, wherein R₂₀ and R₂₁ are independently selected from the group consisting of hydrogen, alkyl, alkenyl, aryl and heteroaryl; and wherein X is selected from the group consisting of CR₂₂R₂₃, N and O, wherein R₂₄ and R₂₅ are independent selected from the group consisting of hydrogen, alkyl, alkenyl, hydroxyl and halo.

### II. Methods of Imaging

In another aspect, methods of imaging are described. Briefly, a method of imaging a target comprises disposing a photoacoustic compound in an environment adjacent to the target and irradiating the photoacoustic compound with one or more wavelengths of electromagnetic radiation. Soundwaves produced by the photoacoustic compound in response to the electromagnetic radiation are detected and transformed into an image. The photoacoustic compound comprises two mutlicyclic ring moieties coupled by a rotationally restricted linkage, and exhibits at least one of a pH dependent absorption spectrum, pH dependent emission spectrum and pH dependent photoacoustic spectrum. In some embodiments, the photoacoustic compound is irradiated with multiple wavelengths of radiation resulting in multiple wavelength dependent images of the target. As described further herein, each of the wavelength dependent images can correspond to the photoacoustic compound at locations of differing pH in the environment. The wavelength dependent images can be overlaid to present a detailed image of the target.

In some embodiments, the target is biological tissue, and the environment is extracellular space in and/or around the tissue. The biological tissue for example, can comprise cancer cells and non-cancer cells. Due to differences in extracellular pH values (pHₑ), the cancer cells can be resolved from the non-cancer cells with the photoacoustic compound. Other biological or physiological environments may be imaged and characterized in this way. Additionally, non-biological environments may also be imaged with photoacoustic compounds described herein based on local pH variations within the environments. Notably, photoacoustic compounds employed in imaging methods can have any composition and/or properties described in Section I above, including structures of Formulas (I) and (II).

These and other embodiments are further illustrated in the follow non-limiting examples.

### Example 1—Photoacoustic Compound

A photoacoustic compound of Formula (Ia) was synthesized and characterized.

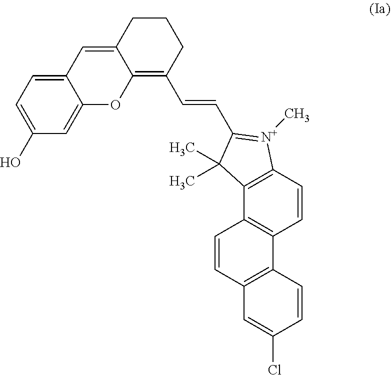 [SMILES: C[N+]1=C(/C=C/C2=C3Oc4cc(O)ccc4C=C3CCC2)C(C)(C)c2c1ccc1c2ccc2cc(Cl)ccc21]

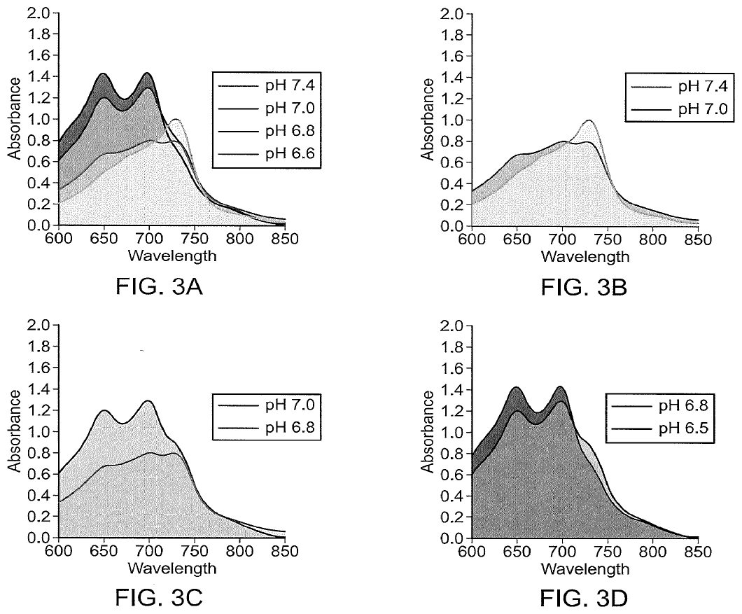

Initial evaluation of compound (Ia) using UV-Vis spectroscopy indicated absorbance spectra modulation corresponding with variable pH PBS buffer solutions (FIGS. 3A-D). Substantial differences were observed between pH 7.4 and pH 7.0 (B), pH 7.0 and 6.8 (C). A small additional peak was observed between pH 6.8 and 6.5 (D). This initial identification of the ability of compound (Ia) to modulate absorbance spectra was solely dependent upon variation in pH as changes in absorbance in various light conditions, various levels of 02, and various cell culture medium (with and without phenol red) (data not shown) were also tested.

The quantum yield of compound (Ia) under acidic (pH 6.0), neutral (7.0), and basic (pH 8.0) buffered solutions were calculated by comparison with rhodamine (ΦR=0.95 in ethanol), where ΦF is the quantum yield. The results are provided in Table I.

TABLE I
Quantum Yield and Spectral Properties

| pH | Absₘₐₓ (nm) | Emₘₐₓ (nm) | ΦF |
|---|---|---|---|
| 6.0 | 652 | 694 | 0.18 |
| 7.0 | 704 | 745 | 0.22 |
| 8.0 | 765 | 810 | 0.23 |

As provided in Table I, absorption and emission maxima blueshift with decreasing pH value.

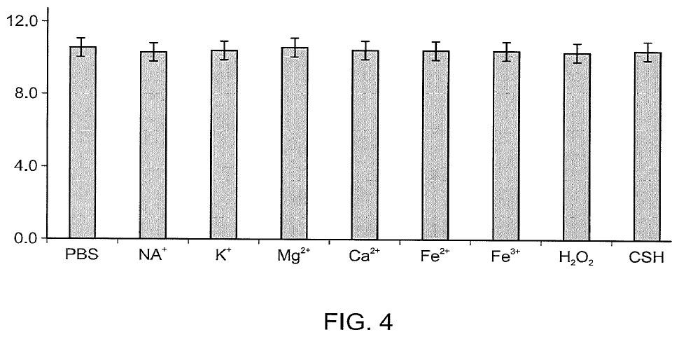

Since compound (Ia) was designed to spectrally alter based upon H⁺ protonation, compound (Ia) was also evaluated in the presence of de-ionized water, phosphate buffer saline, and/or other ions which are present in living organisms (Na⁺, K⁺, Ca²⁺, Fe²⁺, Mg²⁺, Zn²⁺, Fe³⁺ and Fe²⁺ as chloride salts), and other highly reactive cellular products (H₂O₂, glutathione (GSH), cysteine) at pH 7.4. The absorption spectra did not significantly alter in the presence of reactive cellular products or ions other than H⁺ associated with acidic pH (FIG. 4). With the establishment that modulation of spectral absorption corresponded to H⁺ without influence of other ions, compound (Ia) was further evaluated as a potential optoacoustic agent.

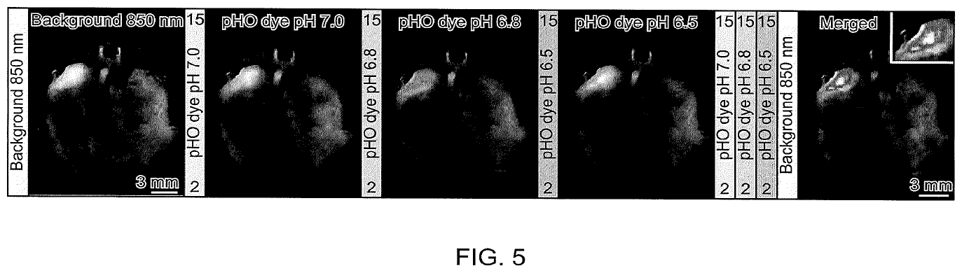

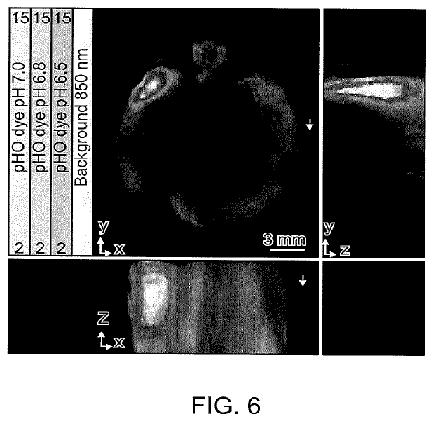

Preliminary evaluation of compound (Ia) accumulation within mice was conducted. A matrigel plug containing surgicel mesh and fibroblast was injected subcutaneously to stimulate non-malignant fibrotic tissue two weeks prior to orthotopic tumor implantation in 3 SCID mice. Mice were anesthesized using 1.5% isoflurane and a gas mixture of 0.9 L medical air and 0.1 L 02 to detect tissue hypoxia. Because areas of tissue hypoxia will also have acidic extracellular pH, tissue hypoxia served as an additional acidic extracellular pH (pHe) control. One week post tumor implantation mice were imaged using MSOT to obtain a baseline prior to iv tail vein injection of compound (Ia) at (100 nM in PBS pH 7.4) in 100 μl total volume. Pancreatic tumor specific accumulation of compound (Ia) was detected after 2 h (FIGS. 5-6). Individual slice at (FIG. 5) and orthogonal image demonstrates 3D accumulation of compound (Ia) (FIG. 6). Based on these results, photoacoustic compound (Ia) exhibits the ability to effectively differentiate cancer and non-cancer cells in MSOT imaging.

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

1. A photoacoustic compound comprising two multicyclic ring moieties coupled by a rotationally restricted linkage, wherein the photoacoustic compound exhibits at least one of pH dependent absorption spectrum, pH dependent emission spectrum and pH dependent photoacoustic spectrum.

2. The photoacoustic compound of claim 1, wherein the photoacoustic compound is zwitterionic.

3. The photoacoustic compound of claim 2, wherein the two multicyclic ring moieties are oppositely charged.

4. The photoacoustic compound of claim 1, wherein the rotationally restricted linkage establishes conjugation between the two multicyclic ring moieties.

5. The photoacoustic compound of claim 1, wherein absorption and/or emission maxima blueshift with decreasing pH value.

6. The photoacoustic compound of claim 1, wherein the absorption, emission and/or photoacoustic spectra change in response to a change in pH value of 0.1 or greater.

7. The photoacoustic compound of claim 1, wherein the absorption, emission and/or photoacoustic spectra change in response to a change in pH value of 0.1 to 0.3.

8. The photoacoustic compound of claim 1, wherein at least one of the multicyclic ring moieties comprises one or more heteroatoms.

9. The photoacoustic compound of claim 1, wherein the absorption, emission and/or photoacoustic spectra are non-responsive to one or more ions selected from the group consisting of alkali metals, alkaline earth metals and transition metals.

10. The photoacoustic compound of claim 1 having Formula (I): 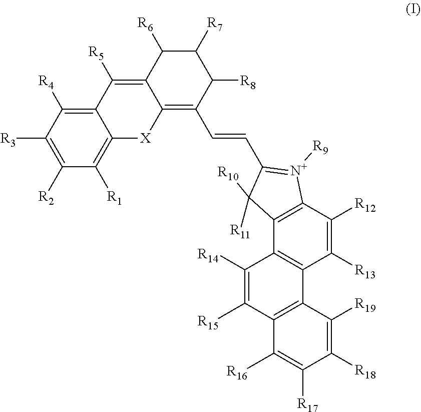
wherein R₁-R₁₉ are independently selected from the group consisting of hydrogen, alkyl, alkenyl, heteroalkyl, cycloalkyl, heterocycloalkyl, aryl, heteroaryl, alkyl-aryl, alkyl-heteroaryl, alkoxy, hydroxyl, halo, and amide, wherein the alkyl, alkenyl, heteroalkyl, cycloalkyl, heterocycloalkyl, aryl, heteroaryl, alkyl-aryl, alkyl-heteroaryl, alkoxy, and amide are optionally substituted with one or more substituents selected from the group consisting of (C₁-C₁₀)-alkyl, (C₁-C₁₀)-alkenyl, heteroalkyl, cycloalkyl, heterocycloalkyl, aryl, heteroaryl, alkoxy, amide, halo, hydroxy, C(O)OR₂₀, and C(O)R₂₁, wherein R₂₀ is selected from the group consisting of hydrogen, alkyl and alkenyl and R₂₁ is selected from the group consisting of hydrogen, alkyl, alkenyl, heteroalkyl, cycloalkyl, heterocycloalkyl, aryl, heteroaryl and NR₂₂R₂₃, wherein R₂₂ and R₂₃ are independently selected from the group consisting of hydrogen, alkyl, alkenyl, aryl and heteroaryl; and wherein X is selected from the group consisting of CR₂₄R₂₅, N and O, wherein R₂₄ and R₂₅ are independent selected from the group consisting of hydrogen, alkyl, alkenyl, hydroxyl and halo.

11. The photoacoustic compound of claim 10, wherein R₁-R₈ and R₁₂-R₁₉ are independently selected from the group consisting of hydrogen, alkyl, cycloalkyl, heteroalkyl, heterocycloalkyl, alkoxy, hydroxyl, and halo and wherein R₉-R₁₁ are independently selected from alkyl and alkenyl.

12. The photoacoustic compound of claim 10 having formula (Ia):

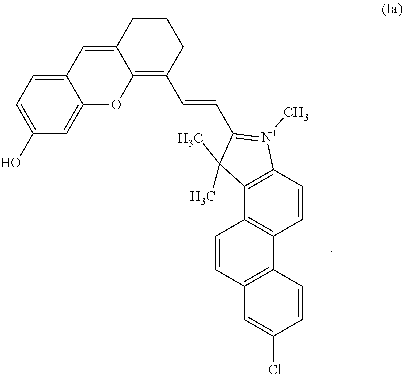 [SMILES: C[N+]1=C(/C=C/C2=C3Oc4cc(O)ccc4C=C3CCC2)C(C)(C)c2c1ccc1c2ccc2cc(Cl)ccc21]

13. The photoacoustic compound of claim 1 having Formula (II): 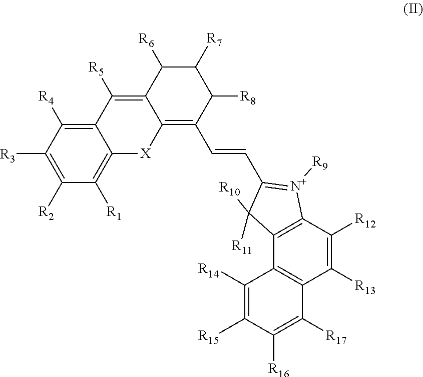
wherein R₁-R₁₇ are independently selected from the group consisting of hydrogen, alkyl, alkenyl, heteroalkyl, cycloalkyl, heterocycloalkyl, aryl, heteroaryl, alkyl-aryl, alkyl-heteroaryl, alkoxy, hydroxyl, halo, and amide, wherein the alkyl, alkenyl, heteroalkyl, cycloalkyl, heterocycloalkyl, aryl, heteroaryl, alkyl-aryl, alkyl-heteroaryl, alkoxy, and amide are optionally substituted with one or more substituents selected from the group consisting of (C₁-C₁₀)-alkyl, heteroalkyl, cycloalkyl, heterocycloalkyl, aryl, heteroaryl, alkoxy, amide, halo, hydroxy, C(O)OR₁₈, and C(O)R₁₉, wherein R₁₈ is selected from the group consisting of hydrogen, alkyl and alkenyl and Rig is selected from the group consisting of hydrogen, alkyl, alkenyl, heteroalkyl, cycloalkyl, heterocycloalkyl, aryl, heteroaryl and NR₂₀R₂₁, wherein R₂₀ and R₂₁ are independently selected from the group consisting of hydrogen, alkyl, alkenyl, aryl and heteroaryl; and wherein X is selected from the group consisting of CR₂₂R₂₃, N and O, wherein R₂₄ and R₂₅ are independent selected from the group consisting of hydrogen, alkyl, alkenyl, hydroxyl and halo.

14. A method of imaging a target comprising:
disposing a photoacoustic compound in an environment adjacent to the target;
irradiating the photoacoustic compound with one or more wavelengths of electromagnetic radiation;
detecting soundwaves produced by the photoacoustic compound in response to the electromagnetic radiation; and
transforming the detected soundwaves into an image, wherein the photoacoustic compound comprises two mutlicyclic ring moieties coupled by a rotationally restricted linkage, and the photoacoustic compound exhibits at least one of a pH dependent absorption spectrum, pH dependent emission spectrum and pH dependent photoacoustic spectrum.

15. The method of claim 14, wherein the photoacoustic compound is zwitterionic.

16. The method of claim 15, wherein the two multicyclic ring moieties are oppositely charged.

17. The method of claim 14, wherein the absorption, emission and/or photoacoustic spectra change in response to a change in pH value of 0.1 or greater.

18. The method of claim 14, wherein the absorption, emission and/or photoacoustic spectra change in response to a change in pH value of 0.1 to 0.3.

19. The method of claim 14, wherein the photoacoustic compound is irradiated with multiple wavelengths of radiation providing multiple wavelength dependent images of the target.

20. The method of claim 19, wherein each of the wavelength dependent images corresponds to the photoacoustic compound at locations of differing pH in the environment.

21. The method of claim 19, wherein the target is biological tissue, and the environment is extracellular space in and/or around the tissue.

22. The method claim 21, wherein the images are superimposed over one another.

23. The method of claim 21, wherein the biological tissue comprises cancer cells and non-cancer cells.

24. The method of claim 23, wherein the cancer cells are resolved from the non-cancer cells by the photoacoustic compound.

25. The method of claim 24, wherein the absorption, emission and/or photoacoustic spectra are non-responsive to one or more ions selected from the group consisting of alkali metals, alkaline earth metals and transition metals.

26. The method of claim 14, wherein the photoacoustic compound is of Formula (I): 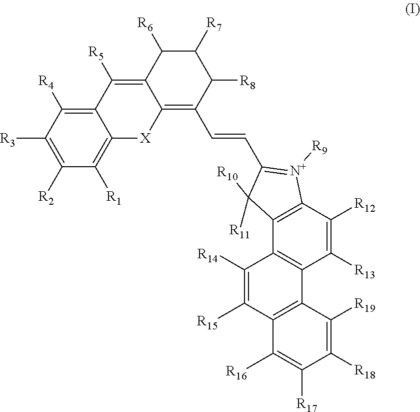
wherein R₁-R₁₉ are independently selected from the group consisting of hydrogen, alkyl, alkenyl, heteroalkyl, cycloalkyl, heterocycloalkyl, aryl, heteroaryl, alkyl-aryl, alkyl-heteroaryl, alkoxy, hydroxyl, halo, and amide, wherein the alkyl, alkenyl, heteroalkyl, cycloalkyl, heterocycloalkyl, aryl, heteroaryl, alkyl-aryl, alkyl-heteroaryl, alkoxy, and amide are optionally substituted with one or more substituents selected from the group consisting of (C₁-C₁₀)-alkyl, (C₁-C₁₀)-alkenyl, heteroalkyl, cycloalkyl, heterocycloalkyl, aryl, heteroaryl, alkoxy, amide, halo, hydroxy, C(O)OR₅, and C(O)R₆, wherein R₅ is selected from the group consisting of hydrogen, alkyl and alkenyl and R₆ is selected from the group consisting of hydrogen, alkyl, alkenyl, heteroalkyl, cycloalkyl, heterocycloalkyl, aryl, heteroaryl and NR₇R₈, wherein R₇ and R₈ are independently selected from the group consisting of hydrogen, alkyl, alkenyl, aryl and heteroaryl; and wherein X is selected from the group consisting of CR₂₄R₂₅, N and O, wherein R₂₄ and R₂₅ are independent selected from the group consisting of hydrogen, alkyl, alkenyl, hydroxyl and halo.

27. The method of claim 26, wherein R₁-R₈ and R₁₂-R₁₉ are independently selected from the group consisting of hydrogen, alkyl, cycloalkyl, heteroalkyl, heterocycloalkyl, alkoxy, hydroxyl, and halo and wherein R₉-R₁₁ are independently selected from alkyl and alkenyl.

28. The method of claim 14, wherein the photoacoustic compound is of Formula (II): 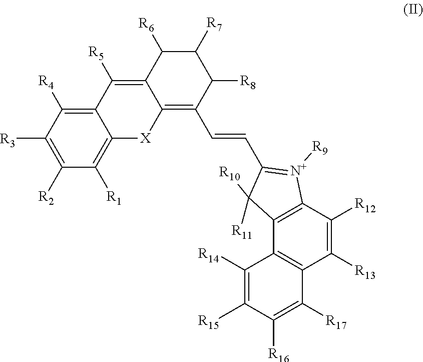
wherein R₁-R₁₇ are independently selected from the group consisting of hydrogen, alkyl, alkenyl, heteroalkyl, cycloalkyl, heterocycloalkyl, aryl, heteroaryl, alkyl-aryl, alkyl-heteroaryl, alkoxy, hydroxyl, halo, and amide, wherein the alkyl, alkenyl, heteroalkyl, cycloalkyl, heterocycloalkyl, aryl, heteroaryl, alkyl-aryl, alkyl-heteroaryl, alkoxy, and amide are optionally substituted with one or more substituents selected from the group consisting of (C₁-C₁₀)-alkyl, (C₁-C₁₀)-alkenyl, heteroalkyl, cycloalkyl, heterocycloalkyl, aryl, heteroaryl, alkoxy, amide, halo, hydroxy, C(O)OR₁₈, and C(O)R₁₉, wherein R₁₈ is selected from the group consisting of hydrogen, alkyl and alkenyl and Rig is selected from the group consisting of hydrogen, alkyl, alkenyl, heteroalkyl, cycloalkyl, heterocycloalkyl, aryl, heteroaryl and NR₂₀R₂₁, wherein R₂₀ and R₂₁ are independently selected from the group consisting of hydrogen, alkyl, alkenyl, aryl and heteroaryl; and wherein X is selected from the group consisting of CR₂₂R₂₃, N and O, wherein R₂₄ and R₂₅ are independent selected from the group consisting of hydrogen, alkyl, alkenyl, hydroxyl and halo.
